(19) United States
(12) Patent Application Publication
(10) Pub. No.: US 2021/0188794 A1
(43) Pub. Date: Jun. 24, 2021

(54) PROCESS FOR PRODUCING 2,5-FURANDICARBOXYLIC ACID DIALKYL ESTER

(71) Applicant: DUPONT INDUSTRIAL BIOSCIENCES USA, LLC, Wilmington, DE (US)

(72) Inventors: PRANIT S. METKAR, Houston, TX (US); Ronnie Ozer, Arden, DE (US); Eric R. Sacia, Wilmington, DE (US)

(21) Appl. No.: 17/188,751

(22) Filed: Mar. 1, 2021

Related U.S. Application Data
(63) Continuation of application No. 16/863,370, filed on Apr. 30, 2020, now abandoned.
Application No. 16/863,370 is a continuation of application No. 15/742,956, filed on Jan. 9, 2018, now abandoned.
(60) Provisional application No. 62/196,790, filed on Jul. 24, 2015.

(51) Int. Cl. C07D 307/68

(52) U.S. Cl. CPC C07D 307/68

(57) ABSTRACT

Disclosed herein are processes for producing 2,5-furandicarboxylic acid dialkyl ester. In one embodiment, the process comprises a) contacting 2,5-furan dicarboxylic acid, excess alcohol, and optionally, a catalyst in a reactor at a temperature in the range of from 50° C. to 325° C. and a pressure in the range of between 1 bar to 140 bar to form a liquid phase composition comprising an ester of 2,5-furan dicarboxylic acid, the alcohol and water; b) lowering the temperature of the liquid phase composition to form a crude crystallized ester of 2,5-furan dicarboxylic acid; c) separating the product of step b) to form a solids phase comprising a purified ester of 2,5-furan dicarboxylic acid and a mother liquor comprising alcohol and water; and d) removing at least a portion of the water from the mother liquor. In one embodiment, the 2,5-furan dicarboxylic acid is contacted with an alcohol source and optionally, a catalyst.

### CROSS-REFERENCE TO RELATED APPLICATION

This application is a Continuation of U.S. application Ser. No. 16/863,370 filed on Apr. 30, 2020, which is a Continuation of U.S. application Ser. No. 15/742,956 filed on Jan. 9, 2018, which is a 371 of International Application No. PCT/US16/43274 filed Jul. 21, 2016, which claims benefit of priority of U.S. Provisional Application No. 62/196,790 filed on Jul. 24, 2015, which is incorporated herein by reference in its entirety.

### FIELD OF THE DISCLOSURE

The present disclosure is directed towards an efficient process for the production of an ester of furan dicarboxylic acid from the esterification of furan dicarboxylic acid (FDCA).

### BACKGROUND OF THE DISCLOSURE

Derivatives of furan are known as potentially being useful in many industries, for example, pharmaceuticals, as fuel components, and as precursors for plastics. It has been disclosed that biomass materials can be used as a raw material to produce furan derivatives that might be useful as intermediates. For example, various sources of biomass can be hydrolyzed to produce pentose and hexose sugars which can then be further processed to form furfural and hydroxymethyl furfural (HMF).

In order to be industrially useful, the furfural and HMF must be efficiently processed to the desired materials. One useful product is furan dicarboxylic acid which can be further processed to form various polymers, including polyesters. Polyesters comprising the furan ring may have useful properties and could provide a replacement or partial replacement for polyesters derived from terephthalic acid. However, there is a continuing need for the efficient production of a furan derivative that can be processed into a furan containing polyester thereby replacing a non-renewable resource, such as terephthalic acid, with a renewable resource.

### SUMMARY OF THE DISCLOSURE

The disclosure relates to a process comprising:
a) contacting 2,5-furan dicarboxylic acid (FDCA), excess alcohol and, optionally, a catalyst in a reactor at a temperature in the range of from 50° C. to 325° C. and a pressure in the range of between 1 bar to 140 bar to form a liquid phase composition comprising an ester of FDCA, the alcohol and water;
b) lowering the temperature of the liquid phase composition to form a crude crystallized ester of FDCA;
c) separating the product of step b) to form a solids phase comprising a purified ester of FDCA and a mother liquor comprising alcohol and water; and
d) removing at least a portion of the water from the mother liquor.
   In one embodiment, the alcohol is methanol.

In another embodiment, the process comprises:
a) contacting FDCA, an alcohol source, optionally, an alcohol, and, optionally, a catalyst in a reactor at a temperature in the range of from 50° C. to 325° C. and a pressure in the range of from 1 bar to 140 bar to form a liquid phase composition comprising an ester of FDCA;
b) lowering the temperature of the liquid phase composition to form a crude crystallized ester of FDCA; and
c) separating the crude crystallized ester of FDCA to form a solids phase comprising a purified ester of FDCA and a mother liquor comprising the alcohol source.

In other embodiments, the process comprises:
a) contacting FDCA, an alcohol source and, optionally, a catalyst in a reactor at a temperature in the range of from 50° to 325° C. and a pressure in the range of from 1 bar to 140 bar to form a liquid composition comprising an ester of FDCA;
b) lowering the temperature of the liquid phase composition to form a crude crystallized ester of FDCA; and
c) separating the product of step b) to form a solids phase comprising a purified ester of FDCA and a mother liquor comprising the alcohol source.
   In some embodiments, the process further comprises a step of recovering at least a portion of the alcohol source from the mother liquor obtained in step c), and optionally recycling the recovered alcohol source to step a).

In other embodiments, the processes further comprise a step of distilling the purified ester of FDCA at a temperature in the range of from 38° C. to 204° C. and a pressure in the range of from 0 bar to 3.5 bar.

In other embodiments, step b) lowering the temperature is conducted in the reactor, in a crystallizing vessel, or in a series of vessels.

In other embodiments, in step b) the temperature is in the range of from −5° C. to 50° C.

In other embodiments, step d) removing at least a portion of the water from the mother liquor is performed by one or more steps of i) distilling the alcohol from the water; ii) passing the mother liquor through an adsorbent bed; iii) passing the mother liquor through molecular sieves; iv) passing the mother liquor through a membrane; or v) passing the mother liquor through a reverse osmosis system.

In other embodiments, the alcohol source is an orthoester, an orthoformate, an acetal, an alkyl carbonate, trialkyl borate a cyclic ether comprising 3 or 4 atoms in the ring or a combination thereof.

### DETAILED DESCRIPTION OF THE DISCLOSURE

The disclosures of all cited patent and non-patent literature are incorporated herein by reference in their entirety.

As used herein, the term “embodiment” or “disclosure” is not meant to be limiting, but applies generally to any of the embodiments defined in the claims or described herein. These terms are used interchangeably herein.

Unless otherwise disclosed, the terms “a” and “an” as used herein are intended to encompass one or more (i.e., at least one) of a referenced feature.

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. In addition, references to the singular may also include the plural (for example, “a” and “an” may refer to one or more) unless the context specifically states otherwise.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

As used herein:

The term “solid acid catalyst” refers to any solid material containing Brönsted and/or Lewis acid sites, and which is substantially undissolved by the reaction medium under ambient conditions.

The phrase “ester of FDCA” means a diester of furan dicarboxylic acid. In some embodiments, the diester of furan dicarboxylic acid is the diester of 2,5-furandicarboxylic acid.

The phrase “alcohol source” means a molecule which, in the presence of water and optionally an acid forms an alcohol.

The acronym FDCA means 2,5-furan dicarboxylic acid.

The acronym FDME means 2,5-furan dicarboxylic acid dimethyl ester.

The acronym FDMME means the monomethyl ester of 2,5-furan dicarboxylic acid.

The acronym FFME means the methyl ester of 5-formylfuran-2-carboxylic acid.

The acronym FFCA means 5-formylfuran-2-carboxylic acid.

The present disclosure relates to efficient processes for producing 2,5-furan dicarboxylic acid dimethyl ester (FDME).

In one embodiment, the process comprises:
a) contacting FDCA, excess alcohol and, optionally, a catalyst in a reactor at a temperature in the range of from 50° C. to 325° C. and a pressure in the range of between 1 bar to 140 bar to form a liquid phase composition comprising an ester of FDCA, the alcohol and water;
b) lowering the temperature of the liquid phase composition to form a crude crystallized ester of FDCA;
c) separating the product of step b) to form a solids phase comprising a purified ester of FDCA and a mother liquor comprising alcohol and water; and
d) removing at least a portion of the water from the mother liquor.

The process comprises a first step of contacting FDCA, excess alcohol and optionally, a catalyst in a reactor. The reactor can be a batch reactor, a continuously stirred tank reactor, a reactive distillation column, a Scheibel column or a plug flow reactor that can be maintained at a temperature in the range of from 50° C. to 325° C. and a pressure in the range of between 1 bar to 140 bar. In some embodiments, the temperature can be in the range of from 75° C. to 325° C., or from 100° C. to 325°, or from 125° C. to 325° C., or from 150° C. to 320° C., or from 160° C. to 315° C., or from 170 to 310° C. In other embodiments, the temperature can be in the range of from 50° C. to 150°, or from 65° C. to 140° C., or from 75° C. to 130° C. In still further embodiments, the temperature can be in the range of from 250° C. to 325° C., or from 260° C. to 320° C., or from 270° C. to 315° C., or from 275° C. to 310° C., or from 280° C. to 310° C. In some embodiments, the pressure can be in the range of from 5 bar to 130 bar, or from 15 bar to 120 bar, or from 20 bar to 120 bar. In other embodiments, the pressure can be in the range of from 1 bar to 5 bar, or 1 bar to 10 bar, or 1 bar to 20 bar.

The alcohol can be an alcohol having in the range of from 1 to 12 carbon atoms, especially alkyl alcohols. Suitable alcohols can include, for example, methanol, ethanol, propanol, butanol, pentanol, hexanol, heptanol, octanol, nonanol, decanol, undecanol, dodecanol or isomers thereof. In some embodiments, the alcohol has in the range of from 1 to 6 carbon atoms, or in the range of from 1 to 4 carbon atoms, or in the range of from 1 to 2 carbon atoms. In some embodiments, the alcohol is methanol and the ester of FDCA is FDME.

In some embodiments, the percentage of FDCA and alcohol that can be fed to the reactor can be expressed as a weight percentage of the FDCA based on the total amount of FDCA and the alcohol. For example, the weight of FDCA can be in the range of from 1 to 70 percent by weight, based on the total weight of the FDCA and the alcohol. Correspondingly, the alcohol can be present at a weight percentage of about 30 to 99 percent by weight, based on the total amount of FDCA and the alcohol. In other embodiments, the FDCA can be present in the range of from 2 to 60 percent, or from 5 to 50 percent, or from 10 to 50 percent or from 15 to 50 percent, or from 20 to 50 percent by weight, wherein all percentages by weight are based on the total amount of FDCA and the alcohol.

In other embodiments, the ratio of alcohol to the FDCA can be expressed in a molar ratio wherein the molar ratio of the alcohol to the FDCA can be in the range of from 2.01:1 to 40:1. In other embodiments, the molar ratio of the alcohol to FDCA can be in the range of from 2.2:1 to 40:1, or 2.5:1 to 40:1, or 3:1 to 40:1, or 4:1 to 40:1, or 8:1 to 40:1, or 10:1 to 40:1, or 15:1 to 40:1, or 20:1 to 40:1, or 25:1 to 40:1, or 30:1 to 40:1.

The contacting step a) can optionally be performed in the presence of a catalyst. If present, the catalyst can be cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst, or a combination thereof. The metal acetates, chlorides, and hydroxides can be used as the hydrated salts. In some embodiments, the catalyst can be cobalt (II) acetate, iron (II) chloride, iron (III) chloride, magnesium (II) acetate, magnesium hydroxide, zinc (II) acetate, or a hydrate thereof. In still further embodiments, the catalyst can be iron (II) chloride, iron (III) chloride. or a combination thereof. In other embodiments, the catalyst can be cobalt acetate. In another embodiment, the catalyst can be sulfuric acid, hydrobromic acid, hydrochloric acid, boric acid, or another suitable Brønsted acid. Combinations of any of the above catalysts may also be useful.

If present, a catalyst can be used at a rate of 0.1 to 5.0 percent by weight, based on the total weight of the FDCA, alcohol and optionally the alcohol source, and the catalyst. In other embodiments, the amount of catalyst present can be in the range of from 0.2 to 4.0, or from 0.5 to 3.0, or from 0.75 to 2.0, or from 1.0 to 1.5 percent by weight, wherein the percentages by weight are based on the total amount of FDCA, methanol and the catalyst.

The catalyst can also be a solid acid catalyst having the thermal stability required to survive reaction conditions. The solid acid catalyst may be supported on at least one catalyst support. Examples of suitable solid acids include without limitation the following categories: 1) heterogeneous heteropolyacids (HPAs) and their salts, 2) natural or synthetic minerals (including both clays and zeolites), such as those containing alumina and/or silica, 3) cation exchange resins, 4) metal oxides, 5) mixed metal oxides, 6) metal salts such as metal sulfides, metal sulfates, metal sulfonates, metal nitrates, metal phosphates, metal phosphonates, metal molybdates, metal tungstates, metal borates or combinations thereof. The metal components of categories 4 to 6 may be selected from elements from Groups 1 through 12 of the Periodic Table of the Elements, as well as aluminum, chromium, tin, titanium, and zirconium. Examples include, without limitation, sulfated zirconia and sulfated titania.

Suitable HPAs include compounds of the general formula XₐM_{b}O_{c}^{q-}, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Nonlimiting examples of salts of HPAs include, for example, lithium, sodium, potassium, cesium, magnesium, barium, copper, gold and gallium, and ammonium salts. Examples of HPAs suitable for the disclosed process include, but are not limited to, tungstosilicic acid (H₄[SiW₁₂O₄₀].xH₂O), tungstophosphoric acid (H₃[PW₁₂O₄₀].xH₂O), molybdophosphoric acid (H₃[PMo₁₂O₄₀].xH₂O), molybdosilicic acid (H₄[SiMo₁₂O₄₀].xH₂O), vanadotungstosilicic acid (H₄₊ₙ[SiVₙW₁₂₋ₙO₄₀].xH₂O), vanadotungstophosphoric acid (H₃₊ₙ[PVₙW₁₂₋ₙO₄₀].xH₂O), vanadomolybdophosphoric acid (H₃₊ₙ[PVₙMo₁₂₋ₙO₄₀].xH₂O), vanadomolybdosilicic acid (H₄₊ₙ[SiVₙMo₁₂₋ₙO₄₀].xH₂O), molybdotungstosilicic acid (H₄[SiMoₙW₁₂₋ₙO₄₀].xH₂O), molybdotungstophosphoric acid (H₃[PMoₙW₁₂₋ₙO₄₀].xH₂O), wherein n in the formulas is an integer from 1 to 11 and x is an integer of 1 or more.

Natural clay minerals are well known in the art and include, without limitation, kaolinite, bentonite, attapulgite, and montmorillonite.

In an embodiment, the solid acid catalyst is a cation exchange resin that is a sulfonic acid functionalized polymer. Suitable cation exchange resins include, but are not limited to the following: styrene divinylbenzene copolymer-based strong cation exchange resins such as AMBERLYST™ and DOWEX® available from Dow Chemicals (Midland, Mich.) (for example, DOWEX® Monosphere M-31, AMBERLYST™ 15, AMBERLITE™ 120); CG resins available from Resintech, Inc. (West Berlin, N.J.); Lewatit resins such as MONOPLUS™ S 100H available from Sybron Chemicals Inc. (Birmingham, N.J.); fluorinated sulfonic acid polymers (these acids are partially or totally fluorinated hydrocarbon polymers containing pendant sulfonic acid groups, which may be partially or totally converted to the salt form) such as NAFION® perfluorinated sulfonic acid polymer, NAFION® Super Acid Catalyst (a bead-form strongly acidic resin which is a copolymer of tetrafluoroethylene and perfluoro-3,6-dioxa-4-methyl-7-octene sulfonyl fluoride, converted to either the proton (H⁺), or the metal salt form) available from DuPont Company (Wilmington, Del.).

In an embodiment, the solid acid catalyst is a supported acid catalyst. The support for the solid acid catalyst can be any solid substance that is inert under the reaction conditions including, but not limited to, oxides such as silica, alumina, titania, sulfated titania, and compounds thereof and combinations thereof; barium sulfate; calcium carbonate; zirconia; carbons, particularly acid washed carbon; and combinations thereof. Acid washed carbon is a carbon that has been washed with an acid, such as nitric acid, sulfuric acid or acetic acid, to remove impurities. The support can be in the form of powder, granules, pellets, or the like. The supported acid catalyst can be prepared by depositing the acid catalyst on the support by any number of methods well known to those skilled in the art of catalysis, such as spraying, soaking or physical mixing, followed by drying, calcination, and if necessary, activation through methods such as reduction or oxidation. The loading of the at least one acid catalyst on the at least one support is in the range of 0.1-20 weight percent based on the combined weights of the at least one acid catalyst and the at least one support. Certain acid catalysts perform better at low loadings such as 0.1-5%, whereas other acid catalysts are more likely to be useful at higher loadings such as 10-20%. In an embodiment, the acid catalyst is an unsupported catalyst having 100% acid catalyst with no support such as, pure zeolites and acidic ion exchange resins.

Examples of supported solid acid catalysts include, but are not limited to, phosphoric acid on silica, NAFION®, a sulfonated perfluorinated polymer, HPAs on silica, sulfated zirconia, and sulfated titania. In the case of NAFION® on silica, a loading of 12.5% is typical of commercial examples.

In another embodiment, the solid acid catalyst comprises a sulfonated divinylbenzene/styrene copolymer, such as AMBERLYST™ 70.

In one embodiment, the solid acid catalyst comprises a sulfonated perfluorinated polymer, such as NAFION® supported on silica (SiO₂).

In one embodiment, the solid acid catalyst comprises natural or synthetic minerals (including both clays and zeolites), such as those containing alumina and/or silica.

Zeolites suitable for use herein can be generally represented by the following formula M_{2/n}O.Al₂O₃.xSiO₂.yH₂O wherein M is a cation of valence n, x is greater than or equal to about 2, and y is a number determined by the porosity and the hydration state of the zeolite, generally from about 2 to about 8. In naturally occurring zeolites, M is principally represented by Na, Ca, K, Mg and Ba in proportions usually reflecting their approximate geochemical abundance. The cations M are loosely bound to the structure and can frequently be completely or partially replaced with other cations by conventional ion exchange.

The zeolite framework structure has corner-linked tetrahedra with Al or Si atoms at centers of the tetrahedra and oxygen atoms at the corners. Such tetrahedra are combined in a well-defined repeating structure comprising various combinations of 4-, 6-, 8-, 10-, and 12-membered rings. The resulting framework structure is a pore network of regular channels and cages that is useful for separation. Pore dimensions are determined by the geometry of the aluminosilicate tetrahedra forming the zeolite channels or cages, with nominal openings of about 0.26 nm for 6-member rings, about 0.40 nm for 8-member rings, about 0.55 nm for 10-member rings, and about 0.74 nm for 12-member rings (these numbers assume the ionic radii for oxygen). Zeolites with the largest pores, being 8-member rings, 10-member rings, and 12-member rings, are frequently considered small, medium and large pore zeolites, respectively.

In a zeolite, the term “silicon to aluminum ratio” or, equivalently, “Si/Al ratio” means the ratio of silicon atoms to aluminum atoms. Pore dimensions are critical to the performance of these materials in catalytic and separation applications, since this characteristic determines whether molecules of certain size can enter and exit the zeolite framework.

In practice, it has been observed that very slight decreases in ring dimensions can effectively hinder or block movement of particular molecular species through the zeolite structure. The effective pore dimensions that control access to the interior of the zeolites are determined not only by the geometric dimensions of the tetrahedra forming the pore opening, but also by the presence or absence of ions in or near the pore. For example, in the case of zeolite type A, access can be restricted by monovalent ions, such as Na⁺ or K⁺, which are situated in or near 8-member ring openings as well as 6-member ring openings. Access can be enhanced by divalent ions, such as Ca²⁺, which are situated only in or near 6-member ring openings. Thus, the potassium and sodium salts of zeolite A exhibit effective pore openings of about 0.3 nm and about 0.4 nm respectively, whereas the calcium salt of zeolite A has an effective pore opening of about 0.5 nm.

The presence or absence of ions in or near the pores, channels and/or cages can also significantly modify the accessible pore volume of the zeolite for sorbing materials. Representative examples of zeolites are (i) small pore zeolites such as NaA (LTA), CaA (LTA), Erionite (ERI), Rho (RHO), ZK-5 (KFI) and chabazite (CHA); (ii) medium pore zeolites such as ZSM-5 (MFI), ZSM-11 (MEL), ZSM-22 (TON), and ZSM-48 (*MRE); and (iii) large pore zeolites such as zeolite beta (BEA), faujasite (FAU), mordenite (MOR), zeolite L (LTL), NaX (FAU), NaY (FAU), DA-Y (FAU) and CaY (FAU). The letters in parentheses give the framework structure type of the zeolite.

Zeolites suitable for use herein include medium or large pore, acidic, hydrophobic zeolites, including without limitation ZSM-5, faujasites, beta, mordenite zeolites or mixtures thereof, having a high silicon to aluminum ratio, such as in the range of 5:1 to 400:1 or 5:1 to 200:1. Medium pore zeolites have a framework structure consisting of 10-membered rings with a pore size of about 0.5-0.6 nm. Large pore zeolites have a framework structure consisting of 12-membered rings with a pore size of about 0.65 to about 0.75 nm. Hydrophobic zeolites generally have Si/Al ratios greater than or equal to about 5, and the hydrophobicity generally increases with increasing Si/Al ratios. Other suitable zeolites include without limitation acidic large pore zeolites such as H—Y with Si/Al in the range of about 2.25 to 5.

After contacting the FDCA with the alcohol for a sufficient period of time at the temperature and pressure conditions given, for example, for one minute to 480 minutes, a liquid phase composition is formed. The liquid phase composition comprises the ester of FDCA, the alcohol and water. In some embodiments, the liquid phase can further comprise the monoalkyl ester of 2,5-furan dicarboxylic acid. The temperature of the liquid phase is then lowered to form a crude crystallized ester of FDCA in step b). The final temperature of the cooled liquid phase composition can be in the range of from −5° C. to 50° C. In other embodiments, the temperature of the liquid phase can be lowered to a temperature in the range of from −5° C. to 40° C., or from −5° C. to 30° C., or from −5° C. to 20° C., or from −5° C. to 10° C. The temperature can be lowered in the same vessel as was used from step a) or it can be a separate vessel or a series of vessels wherein the temperature is gradually lowered in each successive step in the series of vessels. The crude crystallized ester of FDCA can be removed by filtration, centrifugation or a combination thereof after the temperatures reaches the desired final crystallization temperature. In other embodiments, the crude crystallized ester of FDCA can be removed after each step when using a series of crystallization vessels. The separation step forms a solids phase comprising the ester of FDCA and a mother liquor phase comprising the alcohol and water.

The crude crystallized ester of FDCA can then be separated from the liquid layer in a separation step c). The separation step can be done using any of the known solid/liquid separation techniques. For example, the solids phase can be removed by filtration or by centrifugation to give a solids phase comprising a purified ester of FDCA and a mother liquor comprising the alcohol.

The alcohol in the mother liquor phase from the separation step c) can then be purified by removing at least a portion of the water from the mother liquor. The purified alcohol can optionally be reused in step a). The water can be removed by distilling the alcohol from the water, passing the alcohol-water mixture through an absorbent bed, or through molecular sieves, through a membrane, through a reverse osmosis system or through a combination of any one or more of these processes. If the mother liquor is distilled, it can be distilled at a temperature and pressure suitable to separate water from the alcohol. If the alcohol is purified by passing it through molecular sieves, any of the suitable molecular sieves can be used, for example, 3 Å molecular sieves. If the alcohol is purified by passing it through a membrane, any of the suitable membranes can be used.

In some embodiments, the process further comprises a step e) distilling the purified ester of FDCA. The distillation of the purified ester of FDCA can be performed at a pressure in the range of from 0 bar to 3.5 bar and at a temperature in the range of from 38° C. to 204° C. In some embodiments, the distillation step e) is conducted at low pressures, for example, in the range of from less than 1 bar to 0.0001 bar. In other embodiments, the pressure can be in the range of from 0.75 bar to 0.001 bar or from 0.5 bar to 0.01 bar. Purification of the purified ester of FDCA concentrates the unreacted FDCA and the partially esterified FDCA. The FDCA and monoalkyl ester of FDCA can be collected and recycled back into the reaction at step a). Since the distillation of the purified ester of FDCA is a separate process from step d), removing at least a portion of the water from the mother liquor, and requiring different vessels, this step can be performed before, after or concurrently with step c).

In another embodiment, the process comprises:
a) contacting FDCA, an alcohol source, optionally an alcohol, and, optionally, a catalyst in a reactor at a temperature in the range of from 50° C. to 325° C. and a pressure in the range of from 1 bar to 140 bar to form a liquid phase composition comprising an ester of FDCA;
b) lowering the temperature of the liquid phase composition to form a crude crystallized ester of FDCA;
c) separating the crude crystallized ester of FDCA to form a solids phase comprising a purified ester of FDCA and a mother liquor comprising the alcohol source; and
d) optionally, recycling the alcohol source.

In this embodiment, an alcohol source is used in the contacting step a). The alcohol source is a molecule which, in the presence of water and optionally an acid forms an alcohol. In some embodiments, the alcohol source is an acetal, an orthoformate, an alkyl carbonate, a trialkyl borate, a cyclic ether comprising 3 or 4 atoms in the ring, or a combination thereof. Suitable acetals can include, for example, dialkyl acetals, wherein the alkyl portion of the acetal comprises in the range of from 1 to 12 carbon atoms. In some embodiments, the acetal can be 1,1-dimethoxyethane (acetaldehyde dimethyl acetal), 2,2 dimethoxypropane (acetone dimethyl acetal), 1,1-diethoxyethane (acetaldehyde diethyl acetal) or 2,2 diethoxypropane (acetone diethyl acetal). Suitable orthoformates can be, for example, trialkyl orthoformate wherein the alkyl group comprises in the range of from 1 to 12 carbon atoms. In some embodiments, the orthoester is trimethyl orthoformate or triethyl orthoformate. Suitable alkyl carbonates can be dialkyl carbonates wherein the alkyl portion comprises in the range of from 1 to 12 carbon atoms. In some embodiments, the dialkyl carbonate is dimethyl carbonate or diethyl carbonate. Suitable trialkyl borates can be, for example, trialkyl borates wherein the alkyl portion comprises in the range of from 1 to 12 carbon atoms. In some embodiments, the trialkyl borate is trimethyl borate or triethyl borate. A cyclic ether can also be used wherein the cyclic ether has 3 or 4 carbon atoms in the ring. In some embodiments, the cyclic ether is ethylene oxide or oxetane.

In some embodiments, the FDCA can be fed to the reactor at a weight percentage in the range of from 1 to 70 percent of the feed, based on the total weight of the FDCA and the alcohol source or the combination of the alcohol and the alcohol source. Correspondingly, the alcohol source can be present at a weight percentage of about 30 to 99 percent by weight, based on the total weight of FDCA and the alcohol source or the combination of the alcohol source and the alcohol. In other embodiments, the FDCA can be present in the range of from 2 to 60 percent, or from 5 to 50 percent, or from 10 to 50 percent, or from 15 to 50 percent, or from 20 to 50 percent by weight, wherein all percentages by weight are based on the total weight of FDCA and the alcohol source or the combination of the alcohol source and the alcohol.

In the above embodiments, an alcohol or an alcohol source can be used in the contacting step a). In further embodiments, combinations of the alcohol and the alcohol source can also be used. In some embodiments, the percentage by weight of the alcohol can be in the range of from 0.001 percent to 99.999 percent by weight, based on the total weight of the alcohol and the alcohol source. In other embodiments, the alcohol can be present at a percentage by weight in the range of from 1 to 99 percent, or from 5 to 95 percent, or from 10 to 90 percent, or from 20 to 80 percent, or from 30 to 70 percent, or from 40 to 60 percent, wherein the percentages by weight are based on the total weight of the alcohol and the alcohol source.

If a catalyst is present, any of those catalysts shown above can be used.

The step of contacting FDCA, with the alcohol source and optionally, a catalyst can be performed in a reactor. The reactor can be a batch reactor, a continuously stirred tank reactor, a reactive distillation column, a Scheibel column or a plug flow reactor that can be maintained at a temperature in the range of from 50° C. to 325° C. and a pressure in the range of between 1 bar to 140 bar. In some embodiments, the temperature can be in the range of from 75° C. to 325° C., or from 100° C. to 325° C., or from 125° C. to 325° C., or from 150° C. to 320° C., or from 160° C. to 315° C., or from 170 to 310° C. In other embodiments, the temperature can be in the range of from 50° C. to 150° C., or from 65° C. to 140° C., or from 75° C. to 130° C. In still further embodiments, the temperature can be in the range of from 250° C. to 325° C., or from 260° C. to 320° C., or from 270° C. to 315° C., or from 275° C. to 310° C., or from 280° C. to 310° C. In some embodiments, the pressure can be in the range of from 5 bar to 130 bar, or from 15 bar to 120 bar, or from 20 bar to 120 bar. In other embodiments, the pressure can be in the range of from 1 bar to 5 bar, or 1 bar to 10 bar, or 1 bar to 20 bar.

Step b) of this embodiment comprises lowering the temperature to form a crude crystallized ester of FDCA. In this step, the process conditions can be chosen in a similar manner to those process conditions for the embodiment utilizing excess alcohol. After contacting the FDCA with the alcohol for a sufficient period of time, at the temperature and pressure conditions given, for example, for one minute to 240 minutes, a liquid phase composition is formed. The liquid phase composition comprises the ester of FDCA, the alcohol source and water. In some embodiments, the monoalkyl ester of 2,5-furan dicarboxylic acid may be present. The temperature of the liquid phase is then lowered to form a crude crystallized ester of FDCA in step b). The final temperature of the cooled liquid phase composition can be in the range of from −5° C. to 50° C. In other embodiments, the temperature of the liquid phase can be lowered to a temperature in the range of from −5° C. to 40° C., or from −5° C. to 30° C., or from −5° C. to 20° C., or from −5° C. to 10° C. The temperature can be lowered in the same vessel as was used from step a) or it can be a separate vessel or a series of vessels wherein the temperature is gradually lowered in each successive step in the series of vessels.

The crude crystallized ester of FDCA can then be separated from the liquid layer in a separation step c). The separation step can be done using any of the known solid/liquid separation techniques. For example, the solids phase can be removed by filtration or by centrifugation to give a solids phase comprising a purified ester of FDCA and a mother liquor comprising the alcohol source. The mother liquor can comprise any excess alcohol source, if present, and additionally, any of the by-products of the formation of the alcohol for the alcohol source. For example, trimethyl orthoformate, in the presence of water can form methanol and methyl formate. Therefore, methanol and methyl formate can be present in the mother liquor. Other hydrolysis products of the disclosed alcohol sources are well-known in the art and can be present in the mother liquor.

In some embodiments, the process can further comprise step d) wherein the alcohol source is recycled. As used herein, recycling means optionally, purifying the alcohol source and reusing it in the process at step a). In some embodiments, the alcohol source can be purified by distillation. In some embodiments, the impurities that are present in the alcohol source can be the monoester of FDCA or FDCA. If the monoester of FDCA and/or FDCA is present as the impurities, then the alcohol source can be re-used as it is, without a purification step.

The processes disclosed herein can result in an ester of FDCA containing less than 50 parts per million (ppm) of any one of the impurities, for example as determined by HPLC analysis. For example, the ester of FDCA from step e) can contain less than 150 ppm of the alkyl ester of 5-formylfuran-2-carboxylic acid, less than 150 ppm of the monoalkyl ester of 2,5-furan dicarboxylic acid and/or less than 150 ppm FDCA. In other embodiments, the ester of FDCA from step e) can contain less than 25 ppm of the alkyl ester of 5-formylfuran-2-carboxylic acid, less than 25 ppm of the monoalkyl ester of 2,5-furan dicarboxylic acid and/or less than 25 ppm FDCA. In still further embodiments, the ester of FDCA from step e) can contain less than 10 ppm of the alkyl ester of 5-formylfuran-2-carboxylic acid, less than 10 ppm the monoalkyl ester of 2,5-furan dicarboxylic acid and/or less than 10 ppm FDCA.

Non-limiting embodiments of the processes disclosed herein include:

1. A process comprising:
a) contacting FDCA, excess alcohol and, optionally, a catalyst in a reactor at a temperature in the range of from 50° C. to 325° C. and a pressure in the range of between 1 bar to 140 bar to form a liquid phase composition comprising an ester of FDCA, the alcohol and water;
b) lowering the temperature of the liquid phase composition to form a crude crystallized ester of FDCA;
c) separating the product of step b) to form a solids phase comprising a purified ester of FDCA and a mother liquor comprising alcohol and water; and
d) removing at least a portion of the water from the mother liquor.
   2. A process comprising:
a) contacting FDCA, an alcohol source and, optionally, a catalyst in a reactor at a temperature in the range of from 50° to 325° C. and a pressure in the range of from 1 bar to 140 bar to form a liquid composition comprising an ester of FDCA;
b) lowering the temperature of the liquid phase composition to form a crude crystallized ester of FDCA;
c) separating the product of step b) to form a solids phase comprising a purified ester of FDCA and a mother liquor comprising the alcohol source; and
d) optionally, recycling the alcohol source.
   3. The process of embodiment 1 or 2 further comprising step e) distilling the purified ester of FDCA at a temperature in the range of from 38° C. to 204° C. and a pressure in the range of from 0 bar to 3.5 bar.
   4. The process of embodiment 1 or 2 wherein step b) of lowering the temperature is conducted in the reactor, in a crystallizing vessel or in a series of vessels.
   5. The process of embodiment 1 or 2 wherein the lowered temperature of step b) is in the range of from −5° C. to 50° C.
   6. The process of embodiment 1 wherein removal step d) is to distill the alcohol from the water, to pass the mother liquor through an adsorbent bed, to pass the mother liquor through molecular sieves, to pass the mother liquor through a membrane, to pass the mother liquor through a reverse osmosis system or a combination thereof.
   7. The process of embodiment 2 wherein the alcohol source is an orthoester, an orthoformate, an acetal, an alkyl carbonate, trialkyl borate a cyclic ether comprising 3 or 4 atoms in the ring or a combination thereof.

### EXAMPLES

Unless otherwise noted, all materials used herein are available from the Sigma-Aldrich Company, St. Louis, Mo.

### FDCA (Sarchem Labs, 99.0%)

### FDME (Sarchem Labs, 99.0%)

### Methanol (Fisher Scientific, 99.8%)

### N,N-dimethylformamide (DMF) (Sigma Aldrich)

3 Å molecular sieves (Linde 3 Å Molecular Sieves, ⅛″ Extrudates)

### Acetonitrile (Fisher Scientific, A955-1)

### Isopropanol (Fisher Scientific, A4641L1)

Trimethyl orthoformate (Sigma Aldrich, 99%)

### Sulfuric Acid (EMD, 95-98%)

The following abbreviations are used in the examples: “° C.” means degrees Celsius; “wt %” means weight percent; “g” means gram; “min” means minute(s); “μL” means microliter; “ppm” means microgram per gram, “μm” means micrometer; “mL” means milliliter; “mm” means millimeter and “mL/min” means milliliter per minute; “v/v” means volume for volume; “FFCA” means 5-formyl-2-furancarboxylic acid, “FDCA” means 2,5-furandicarboxylic acid, “FDME” means 2,5-furandicarboxylic acid dimethyl ester, “FDMME” means monomethyl ester of 2,5-furan dicarboxylic acid; “TFA” means trifluoroacetic acid.

### Test/General Methods

### HPLC Analysis

HPLC analysis was used as one means to measure the FDCA, FDMME, and FDME contents of the product mixture. An Agilent 1200 series HPLC equipped with a ZORBAX® SB-aq column (4.6 mm×250 mm, 5 μm) and photodiode array detector was used for the analysis of the reaction samples. The wavelengths used to monitor the reaction were 254 and 280 nm. The HPLC separation of FDME, FDCA and FDMME was achieved using a gradient method with a 1.0 mL/min flow rate combining two mobile phases: Mobile Phase A: 0.5% v/v TFA in water and Mobile Phase B: acetonitrile. The column was held at 60° C. and 2 μL injections of samples were performed. Analyzed samples were diluted to <0.1 wt % for components of interest in either a 50:50 (v/v) acetonitrile/isopropanol solvent or a 2:1 (v/v) dimethyl formamide/isopropanol solvent. The solvent composition and flow rates used for the gradient method are given in Table 1 with linear changes occurring over the corresponding step whenever the composition changes.

TABLE 1
Gradient program for HPLC

| | Start | Volume % Mobile | Volume % Mobile |
|---|---|---|---|
| | time | Phase B, at | Phase B, at |
| Step | (min) | Beginning of Step | End of Step |
| 1 | 0.0 | 0 | 0 |
| 2 | 6.0 | 0 | 80 |
| 3 | 20.0 | 80 | 80 |
| 4 | 25.0 | 80 | 0 |
| 5 | 25.1 | 0 | 0 |
| 6 | 30.0 | 0 | 0 |

Retention times were obtained by injecting analytical standards of each component onto the HPLC. The amount of the analyte in weight percent was typically determined by injection of two or more injections from a given prepared solution and averaging the area measured for the component using the OpenLAB CDS C.01.05 software. The solution analyzed by HPLC was generated by dilution of a measured mass of the reaction sample with a quantified mass of solvent. Calibration of response factors for analytes of interest was performed in the same solvent system as used for reaction analysis. Quantification was performed by comparing the areas determined in the OpenLAB software to a linear external calibration curve at five or more starting material concentrations. Typical R² values for the fit of such linear calibration curves were in excess of 0.9997.

While the presented HPLC method was used for this analysis, it should be understood that any HPLC method that can discriminate between products, starting materials, intermediates, impurities, and solvent can be used for this analysis. It should also be understood that while HPLC was used as a method of analysis in this work, other techniques such as gas chromatography could also be optionally used for quantification when employing appropriate derivatization and calibration as necessary.

### Example 1

### 1 L Batch Esterification of FDCA to FDME at 220° C.

The esterification of FDCA was carried out in a 1 L Parr Zirconium reactor model 4520. FDCA (69.4 g) and 279.6 g methanol were added to the reactor. The reactor was stirred at 400 rpm by an electric stirrer, and heated by an electric band heater around the bottom of the vessel that was insulated with jacketing. The reactor was purged 3 times with nitrogen at 100 psi. At room temperature, 100 psi of N₂ was introduced in the reactor head. The reactor was then heated to an internal temperature of 220° C. and both the temperature and pressure were monitored. During the experiment, liquid samples were taken from the bottom of the vessel at the following times: 0 minutes (when reactor reaches 220° C.), 15 minutes, 30 minutes, 60 minutes, 120 minutes, 240 minutes, 360 minutes, and 480 minutes. After 8 hr, heat was turned off and the reactor was allowed to cool to room temperature. After the reactor temperature cooled to room temperature, pressure was released and reactor opened. The reactor contents were removed and transferred to an aluminum pan and the reactor was rinsed with methanol. Solids and liquid samples taken during experiment were dried overnight in air, and then dried for at least 4 hours at 80° C. in a vacuum oven. The dried solids were then analyzed by HPLC; results are presented in Table 2.

TABLE 2
FDCA, FDME, and FDMME solids concentrations
over 8 hours at 220° C.

| | | FDCA | FDME | FDMME |
|---|---|---|---|---|
| | Time | (solids | (solids | (solids |
| Sample | (min) | mass %) | mass %) | mass %) |
| 1.1 | 0 | 8.85 | 48.80 | 42.35 |
| 1.2 | 15 | 3.24 | 67.86 | 28.90 |
| 1.3 | 30 | 1.03 | 79.70 | 19.27 |
| 1.4 | 60 | 0.00 | 87.31 | 12.69 |
| 1.5 | 120 | 0.00 | 90.32 | 9.68 |
| 1.6 | 240 | 0.00 | 87.94 | 12.06 |
| 1.7 | 360 | 0.00 | 87.83 | 12.17 |
| 1.8 | 480 | 0.00 | 88.66 | 11.34 |

The FDME solids mass percent increased until the 120 minute time point. After this the reaction was determined to be equilibrium limited and the FDME solids mass percent stays centered around 88%.

### Example 2

### Esterification Followed by Water Removal with Molecular Sieves

The esterification of FDCA was carried out in a 75 mL Parr reactor model 5050 equipped with an IKA RCT Basic hotplate stirrer. 8 g FDCA, 32 g methanol and TFE stir bar were added to the reactor. The reactor was placed in an aluminum block and kept insulated. The reactor was then purged a minimum of 5 times with nitrogen. At room temperature, 300 psi of N₂ was introduced in the reactor head. The reactor was heated to a temperature of 200° C. and both the temperature and pressure were monitored. After 4 hr, the heat was turned off and the reactor allowed to cool down. When the reactor cooled room temperature, pressure was released and reactor opened. At the end of this reaction, reactor contents (containing mainly FDME product) were removed and filtered. The solids (Sample 2.1) were separated from the mother liquor during filtration and were analyzed using the HPLC method described. The original mother liquor (Sample 2.2) was analyzed for its water content using a Karl Fischer titrator (Mettler Toledo DL-31). After the Karl-Fischer analysis, 10.6 g mother liquor was separated and added to a sealed container which contained 5.3 g of 3 Å molecular sieves (in the form of ⅛″ extrudates) and a magnetic stirrer. The molecular sieves were activated before use. The activation procedure involved heating the molecular sieves from room temperature to 525° C. at 10° C./min, then from 525° C. to 540° C. at 2° C./min, then from 540° C. to 550° C. at 1° C./min, followed by a 10 hour hold at 550° C. before cooling to 110° C. The sealed contents were stirred at room temperature for 1 hr and then filtered. After filtration, the mother liquor was then separated from the molecular sieves. The mother liquor obtained after molecular sieve treatment (Sample 2.3) was then analyzed for water content using the Karl Fischer titrator.

TABLE 3
HPLC analysis of solid products obtained
at the end of esterification reaction

| Sample | FDCA (wt. %) | FDMME (wt. %) | FDME (wt. %) |
|---|---|---|---|
| 2.1 | 0.23 | 7.08 | 92.69 |

TABLE 4
Results obtained for water content in a mother liquor before
and after the treatment with 3 Å mol sieves.

| Sample | Description | Water content (%) |
|---|---|---|
| 2.2 | Mother liquor obtained after | 7.88 |
| | the esterification reaction | |
| 2.3 | Mother liquor treated with | 3.88 |
| | molecular sieves | |

These results indicate that the water content in the methanol mother liquor from the esterification process can be reduced significantly by using molecular sieves. The impact of contact time with the molecular sieves and the amount of molecular sieves used is expected to have significance on altering the amount of water removal from the methanol mother liquor.

### Example 3

### Esterification of FDCA to FDME as a Function of Temperature

The esterification of FDCA solids was carried out in a series of 4 mL batch tube reactors immersed in a Techne sand bath which was fluidized at the temperature of interest. Each reactor was loaded with 0.1 g FDCA, 1.5 g FDME and 500 microLiters of methanol in air and optionally an acid catalyst and sealed with a Swagelok tubing plug (316 Stainless Steel, pressure rating 3300 psig (228.6 bar)). The sealed reactor was then inserted into the sand bath and after the desired time the reactor was removed and immersed in cold water to quench the reaction. After the reactor had cooled to room temperature, any remaining pressure was released and reactor opened. The reactor contents were removed and transferred to an aluminum pan and the reactor was rinsed with methanol. The solids were dried overnight in air in an aluminum pan, and then for at least 4 hours at 80° C. in a vacuum oven. The dried solids were analyzed by HPLC using the analysis described in the Test Methods section.

For the experiments in Table 5 below, each temperature and time shown was completed twice. The reported wt % values of FDCA, FDMME and FDME in Table 5 are an average of the two measurements. The starting composition for each tube was 5 wt % FDCA, 75 wt % FDME and 20 wt % methanol.

TABLE 5
Results obtained after the analysis of solids collected at the
end of Sand Bath tests at 200, 230 and 270° C. are shown below.

| | Temp | Duration | | | |
|---|---|---|---|---|---|
| Sample | (° C.) | (min) | FDCA % | FDMME % | FDME % |
| 3.1 | 200 | 15 | 5.96 | 2.39 | 91.65 |
| 3.2 | 200 | 30 | 4.42 | 2.34 | 93.24 |
| 3.3 | 200 | 60 | 2.60 | 7.17 | 90.22 |
| 3.4 | 230 | 15 | 2.36 | 5.19 | 92.45 |
| 3.5 | 230 | 30 | 0.29 | 5.39 | 94.32 |
| 3.6 | 230 | 60 | 0.37 | 7.19 | 92.44 |
| 3.7 | 270 | 15 | 0.22 | 5.79 | 93.99 |
| 3.8 | 270 | 30 | 0.18 | 7.17 | 92.65 |
| 3.9 | 270 | 60 | 0.32 | 11.41 | 88.27 |

The results indicate the reaction rate increasing as a function of temperature, exhibited by the disappearance of FDCA and the production of FDMME. Table 5 shows that the conversion of FDCA was nearly complete at 15 minutes at 270° C., at 30 minutes at 230° C. and far from complete at 200° C. HPLC analysis reveals only the three indicated products in the dried solids, suggesting reaction stability at these temperatures.

### Example 4

### Stability of FDME at High Temperature

The stability of FDME was studied in the presence of FDCA and methanol. This study was carried out in a 75 mL Parr reactor model 5050 equipped with an IKA RCT Basic hotplate stirrer. 24 g FDME, 0.5 g FDCA, 0.5 g methanol and a TFE stir bar were added to the reactor. The reactor was placed in an aluminum block and was kept insulated. The reactor was then purged a minimum of 5 times with nitrogen. At room temperature, 300 psi of N₂ was introduced in the reactor head. The reactor was then heated to a desired temperature and both the temperature and pressure are monitored. After 1 hr, heat was turned off and the reactor was allowed to cool down. After the reactor temperature dropped down to room temperature, pressure was released and the reactor was opened. The reactor contents were removed and analyzed using different methods as stated below. This experiment was carried out at two different temperatures (270° C. & 300° C.) starting with a fresh sample of FDME (mixed with FDCA & methanol). The starting/original/control FDME sample is labeled as Sample A. The sample obtained at the end of 270° C. run is labeled as Sample 4.1. The sample obtained at the end of 300° C. run is labeled as Sample 4.2.

HPLC analysis and color analysis were used to measure the stability of FDME heated at high temperature. The solids collected at the end of above heating cycle were analyzed for FDME, FDMME, and FDCA. The results are summarized in Table 6.

TABLE 6
Results obtained after the analysis of solids
collected at the end of stability tests

| | Temp | | | Wt. % | Wt. % | Wt. % | Humins |
|---|---|---|---|---|---|---|---|
| Sample | (° C.) | L* | b* | FDME | FDMME | FDCA | (ppm) |
| A | — | 99.21 | 0.18 | 99.43 | 0.57 | 0 | <10 |
| 4.1 | 270 | 99.58 | 0.64 | 92.41 | 6.96 | 0.1 | <10 |
| 4.2 | 300 | 98.84 | 1.03 | 94.5 | 5.3 | 0.1 | <10 |

It is observed that the FDME is stable under the conditions tested above. The mass balance for furanics (FDME, FDMME, FDCA, etc.) was found to be very close to ˜100% which shows that there was no loss of furanics under the condition tested above. The L* and b* values are very close to that of starting FDME material. The amount of humins formed during these experiment was below the detection limit of the SEC method (10 ppm). This indicates that the degradation of furanics to humins was not observed. Overall, FDME was found to be very stable under the conditions investigated.

### Example 5

### 300 mL Batch Esterification of FDCA to FDME at 220° C.

The esterification of FDCA was carried out in a model 452HC 300 mL Parr Titanium reactor. FDCA (20.0 g), trimethyl orthoformate (36.3 g), and methanol (EMD DriSolv, >99.8%, <50 ppm H₂O) (46.6 g) were added to the reactor. Additionally, for sample 5.2, 0.20 g of sulfuric acid was also added to the reactor. The reactor was sealed and purged three times with nitrogen at 100 psig. At the beginning of the run and at room temperature, 100 psig of N₂ was introduced in the reactor head. The reactor was stirred at 400 RPM by a mag-drive stirrer and was heated by an electric heating mantle around the bottom of the vessel. The reactor was then heated to an internal temperature of 220° C. and both the temperature and pressure were monitored. The reactor was held at temperature for the time period shown in Table 7, after which the heat was turned off, and the reactor was allowed to cool to room temperature. After the reactor cooled to room temperature, pressure was released and the reactor was opened. The reactor contents were removed and transferred to a 250 mL glass bottle and cooled to approximately 0° C. in an ice bath prior to filtering the mixture. The reactor was rinsed with methanol to recover any remaining material. The filtered solids were dried for 4 hours at 50° C. in a vacuum oven at a pressure between −20 and −25 inches of mercury under a continuous flow of N₂. The dried solids, mother liquor from filtration, and reactor methanol wash were then analyzed by HPLC. The normalized product breakdown on the basis of total amount of FDCA, FDMME, and FDME is shown in Table 7.

TABLE 7
FDCA, FDME, and FDMME reaction product
composition using an alcohol source

| | Sulfuric | Time | FDCA | FDME | FDMME |
|---|---|---|---|---|---|
| Sample | Acid (wt %) | (min) | (mass %) | (mass %) | (mass %) |
| 5.1 | 0 | 60 | 0.3 | 99.4 | 0.3 |
| 5.2 | 0.2 | 20 | 0.9 | 85.9 | 13.2 |

The FDME mass percent increased well beyond that shown in Example 1 in Sample 5.1 due to the inclusion of an alcohol source. The alcohol source, in this case trimethyl orthoformate, reacts with water to form methanol. The removal of water in the reaction increases yields to FDME by Le Chatelier's principle. Further, the inclusion of a sulfuric acid catalyst in Sample 5.2 allows for mass percentages of FDME approximately equivalent to those measured at 60 minutes in Sample 1.4 after only 20 minutes of reaction, demonstrating the potential impact of a catalyst on reaction rate.

What is claimed is:

1. A process comprising:
a) contacting 2,5-furan dicarboxylic acid, excess alcohol and, optionally, a catalyst in a reactor at a temperature in the range of from 50° C. to 325° C. and a pressure in the range of between 1 bar to 140 bar to form a liquid phase composition comprising an ester of 2,5-furan dicarboxylic acid, the alcohol and water;
b) lowering the temperature of the liquid phase composition to form a crude crystallized ester of 2,5-furan dicarboxylic acid;
c) separating the product of step b) to form a solids phase comprising a purified ester of 2,5-furan dicarboxylic acid and a mother liquor comprising alcohol and water; and
d) removing at least a portion of the water from the mother liquor.

2. The process of claim 1, further comprising step e) distilling the purified ester of 2,5-furan dicarboxylic acid at a temperature in the range of from 38° C. to 204° C. and a pressure in the range of from 0 bar to 3.5 bar.

3. The process of claim 1 wherein step b) lowering the temperature is conducted in the reactor, in a crystallizing vessel, or in a series of vessels.

4. The process of claim 1, wherein in step b) the temperature is in the range of from −5° C. to 50° C.

5. The process of claim 1, wherein step d) is performed by one or more steps of
i) distilling the alcohol from the water;
ii) passing the mother liquor through an adsorbent bed;
iii) passing the mother liquor through molecular sieves;
iv) passing the mother liquor through a membrane; or
v) passing the mother liquor through a reverse osmosis system.

6. The process of claim 1, wherein the alcohol is methanol.

7. A process comprising:
a) contacting 2,5-furan dicarboxylic acid, an alcohol source and, optionally, a catalyst in a reactor at a temperature in the range of from 50° to 325° C. and a pressure in the range of from 1 bar to 140 bar to form a liquid composition comprising an ester of 2,5-furan dicarboxylic acid;
b) lowering the temperature of the liquid phase composition to form a crude crystallized ester of 2,5-furan dicarboxylic acid; and
c) separating the product of step b) to form a solids phase comprising a purified ester of 2,5-furan dicarboxylic acid and a mother liquor comprising the alcohol source.

8. The process of claim 7, further comprising step d) distilling the purified ester of 2,5-furan dicarboxylic acid at a temperature in the range of from 38° C. to 204° C. and a pressure in the range of from 0 bar to 3.5 bar.

9. The process of claim 7, wherein step b) lowering the temperature is conducted in the reactor, in a crystallizing vessel, or in a series of vessels.

10. The process of claim 7, wherein in step b) the temperature is in the range of from −5° C. to 50° C.

11. The process of claim 7, wherein the alcohol source is an orthoester, an orthoformate, an acetal, an alkyl carbonate, a trialkyl borate, a cyclic ether comprising 3 or 4 atoms in the ring, or a combination thereof.

12. The process of claim 7, further comprising a step of recovering at least a portion of the alcohol source from the mother liquor obtained in step c), and optionally recycling the recovered alcohol source to step a).
